(19) United States
(12) Patent Application Publication
(10) Pub. No.: US 2010/0278836 A1
(43) Pub. Date: Nov. 4, 2010

(54) DIAGNOSTIC AND THERAPEUTIC METHODS AND COMPOSITIONS FOR CARDIOVASCULAR DISEASE

(75) Inventors: Hans Grönlund, Lindingo (SE); Johan Frostegard, Stockholm (SE); Ingrid Dahlbom, Uppsala (SE)

Correspondence Address: FULBRIGHT & JAWORSKI L.L.P., 600 CONGRESS AVE., SUITE 2400, AUSTIN, TX, 78701, US

(73) Assignee: ATHERA BIOTECHNOLOGIES AB, Stockholm (SE)

(21) Appl. No.: 12/740,749

(22) PCT Filed: Oct. 29, 2008
(86) PCT No.: PCT/GB2008/003661

§ 371 Date: Jul. 15, 2010

Related U.S. Application Data
(60) Provisional application No. 60/983,880, filed on Oct. 30, 2007.

(51) Int. Cl. A61K 39/395; A61K 31/685; A61P 9/00; G01N 33/53

(52) U.S. Cl. 424/152.1; 514/77; 436/513

(57) ABSTRACT

Low levels of antibodies reactive with a PAF-conjugate are related to an increased risk of developing cardiovascular diseases. Thus, compositions and methods of diagnosis and therapy for cardiovascular diseases are provided.

### BACKGROUND OF THE INVENTION

I. Field of the Invention

This invention relates to the field of prevention, treatment and risk assessment for atherosclerosis and cardiovascular diseases.

II. Description of Related Art

Atherosclerosis is a chronic disease that causes a thickening of the innermost layer (the intima) of large and medium-sized arteries. It decreases blood flow and may cause ischemia and tissue destruction in organs supplied by the affected vessel. Atherosclerosis is the major cause of cardiovascular disease including myocardial infarction, stroke and peripheral artery disease. It is the major cause of death in the western world and is predicted to become the leading cause of death in the entire world within two decades.

The disease is initiated by accumulation of lipoproteins, primarily low-density lipoprotein (LDL), in the extracellular matrix of the vessel. These LDL particles aggregate and undergo oxidative modification. Oxidized LDL is pro-inflammatory, toxic and causes vascular injury. Atherosclerosis represents in many respects a response to this injury including inflammation and fibrosis.

In 1989 Palinski W et al. identified circulating autoantibodies against oxidized LDL in humans (Low density lipoprotein undergoes oxidative modification in vivo. Proc Natl Acad Sci USA. 1989, 86:1372-6).

This observation suggested that atherosclerosis may be an autoimmune disease caused by immune reactions against oxidized lipoproteins.

At this time several laboratories began searching for associations between antibody titers against oxidized LDL and cardiovascular disease. However, the picture that emerged from these studies was far from clear. Antibodies existed against a large number of different epitopes in oxidized LDL, but the structure of these epitopes was unknown. The term “oxidized LDL antibodies” thus referred to a mixture of antibodies of different specificities, rather than to antibodies of a single specificity.

It is well established that there is an ongoing inflammation in the atherosclerotic lesions, characterized by activation of immune competent cells and production of inflammatory cytokines. Established risk factors like hypertension, blood lipids, diabetes and smoking are likely to promote this inflammatory reaction, but the mechanism by which this occurs are not well characterized and different non-mutually exclusive possibilities exist. Several different autoantigens that could elicit this immune reactivity have been proposed, including oxidized low density lipoprotein (oxLDL) and heat shock proteins (HSP) (Binder C J et al. Innate and acquired immunity in atherogenesis. Nat. Med. 2002, 8:1218-26; Frostegard J. Autoimmunity, oxidized LDL and cardiovascular disease. Autoimmun Rev. 2002, 1:233-7). Available data on the role of immune reactions in atherosclerosis indicate a complex relationship. One example of this is immunization in animal models to influence atherogenesis. When HSP is used, atherosclerosis increases but decreases when oxLDL is the antigen (Palinski W et al. Immunization of low density lipoprotein (LDL) receptor-deficient rabbits with homologous malondialdehyde-modified LDL reduces atherogenesis. Proc Natl Acad Sci USA. 1995, 92:821-5; Xu Q et al. Induction of arteriosclerosis in normocholesterolemic rabbits by immunization with heat shock protein 65. Arterioscler Thromb. 1992, 12:789-99).

The role of antibodies directed to oxLDL (anti-OxLDL) in human disease appears to be complex. In humans, it has previously been demonstrated that anti-OxLDL is higher in healthy controls than in men with borderline hypertension, an example of early cardiovascular disease (Wu R et al. Autoantibodies to OxLDL are decreased in individuals with borderline hypertension. Hypertension 1999, 33:53-59).

On the other hand, several authors have reported that anti-OxLDL are raised in human cardiovascular diseases (CVD), especially at later stages (Bergmark C et al. Patients with early-onset peripheral vascular disease have increased levels of autoantibodies against oxidized LDL. Arterioscler Thromb Vasc Biol. 1995, 15:441-5; Salonen J T et al. Autoantibody against oxidised LDL and progression of carotid atherosclerosis. Lancet 1992, 339:883-887). One example is systemic lupus erythematosus (SLE), an autoimmune disease associated with a very high risk of CVD. SLE-patients with a history of CVD had clearly raised anti-OxLDL-levels (Svenungsson E et al. Risk factors for cardiovascular disease in systemic lupus erythematosus. Circulation 2001, 104:1887-93). These to some extent contradictory results may depend on different methods and stages of LDL-oxidation, yielding differences in antigenicity. It is also likely that disease stage and risk factor profile are related to antibody levels.

Oxidized low density lipoprotein (oxLDL) itself has many proinflammatory properties including activation of T cells 1, monocytes/macrophages and endothelial cells (Berliner J A et al. Minimally modified low density lipoprotein stimulates monocyte endothelial interactions. J Clin Invest. 1990, 85:1260-6; Frostegard J et al. Oxidized low density lipoprotein induces differentiation and adhesion of human monocytes and the monocytic cell line U937. Proc Natl Acad Sci USA. 1990, 87:904-8; Frostegard J et al. Biologically modified LDL increases the adhesive properties of endothelial cells. Atherosclerosis. 1991, 90:119-26).

However, it should be noted that oxLDL may also ameliorate acute inflammatory reactions and instead promote a more low-grade chronic inflammation as that seen in atherosclerosis. It is interesting to note that many biological effects of oxLDL are caused by platelet activating factor (PAF)-like lipids in oxLDL (Frostegard J et al. Platelet-activating factor and oxidized LDL induce immune activation by a common mechanism. Arterioscler Thromb Vasc Biol. 1997, 17:963-8; Heery J M et al. Oxidatively modified LDL contains phospholipids with platelet-activating factor-like activity and stimulates the growth of smooth muscle cells. J Clin Invest. 1995, 96:2322-30; Subbanagounder G et al. Evidence that phospholipid oxidation products and/or platelet-activating factor play an important role in early atherogenesis: in vitro and in vivo inhibition by WEB 2086. Circ Res. 1999, 85:311-8).

Platelet activating factor (PAF) is a phospholipid inflammatory mediator that is synthesized by a variety of cells, including monocytes and endothelial cells. During oxidation of LDL, PAF-like lipids are produced. PAF may therefore be of importance in pathological processes in the vascular wall like atherosclerosis and hypertension. In a previous report, the existence of antibodies to PAF were described in individuals with phospholipid antibody syndrome (Barquinero J et al. Antibodies against platelet-activating factor in patients with antiphospholipid antibodies. Lupus 1994, 3: 55-58).

Tektonidou M G et al. (Clinical importance of antibodies against platelet activating factor in antiphospholipid syndrome manifestations. Eur J Clin Invest. 2000, 30:646-52) reported that anti-PAF antibodies are common in APS and SLE and comprise an independent factor for the development of thrombosis.

WO 00/02046 describes that elevated concentrations of antibodies to PAF is correlated to borderline hypertension and metabolic syndrome, i.e. early cardiovascular disease. Elevated concentrations of antibodies to PAF is shown to be connected to increased risk of developing early atherosclerosis.

Wu et al. (Antibodies to platelet-activating factor are associated with borderline hypertension, early atherosclerosis and the metabolic syndrome. J Intern Med. 1999 246: 389-397) further suggested that anti-PAF antibodies may reflect early vascular changes and thus serve as novel markers for disease, and that they may also be pathogenic, by eliciting an inflammatory reaction in the vascular wall.

WO 2005/100405 describes methods to determine the presence or absence of antibodies, for example IgM or IgG antibodies, against phosphorylcholine (PC) which are related to an increased or decreased risk of developing ischemic cardiovascular diseases, and further suggest the use of PC-conjugates or antibodies directed to PC-conjugates in the treatment or prevention of atherosclerosis.

### SUMMARY OF THE INVENTION

A first aspect of the invention provides the use of at least one PAF derivative, one PAF conjugate, or an antibody preparation, for example a monoclonal antibody, with reactivity to a PAF conjugate, in the manufacture of a medicament for immunization and prophylaxis, prevention or treatment of mammals, including humans, against cardiovascular disease, such as atherosclerosis or an atherosclerotic related disease. The medicament is intended to provide immunization having immunogenic or therapeutic properties against cardiovascular disease.

A second aspect of the invention provides a method for immunization and treatment of a mammal, including a human, against cardiovascular disease, such as atherosclerosis or an atherosclerotic related disease, the method comprising the step of administering to the mammal a pharmaceutical composition comprising at least one PAF derivative, one PAF conjugate, or an antibody preparation, for example a monoclonal antibody, with reactivity to a PAF conjugate. The pharmaceutical composition is intended to provide immunization having immunogenic or therapeutic properties against atherosclerosis.

A third aspect of the invention provides the use of one or more of the PAF derivatives and PAF conjugates as defined in relation to the preceding aspects of the invention, in the manufacture of a pharmaceutical composition, optionally in combination with an adjuvant, for immunotherapy or therapy for the prevention, prophylaxis and/or treatment of cardiovascular diseases.

A fourth aspect of the invention provides a method of prophylactic or therapeutic treatment of a mammal, which may be a human being, suffering from atherosclerosis or facing the risk of developing cardiovascular disease, whereby a therapeutically effective amount of at least one PAF derivative, one PAF conjugate or an antibody preparation, for example a monoclonal antibody, with reactivity to a PAF conjugate is administered.

A fifth aspect of the invention provides a method of diagnosing the presence or absence of antibodies, for example IgM, IgG or IgA antibodies, related to increased or decreased risk of developing cardiovascular diseases, using a PAF conjugate.

A sixth aspect of the invention provides the use of a PAF conjugate in a method for assessing a patient's risk of developing or progression of cardiovascular disease in which the patient's levels of antibodies, for example IgM, IgG or IgA antibodies, with reactivity to the PAF conjugate are assessed.

### DETAILED DESCRIPTION OF THE INVENTION

The present inventors have surprisingly found that low levels of antibodies reactive with a PAF-conjugate is related to an increased risk of developing cardiovascular diseases. This is contrary to what has been reported by the prior art, where high levels of antibodies to PAF has been suggested to be related to an increased risk of developing cardiovascular diseases

The invention relates to pharmaceutical compositions comprising a PAF derivative, a PAF conjugate, or an antibody preparation, for example a monoclonal antibody, with reactivity to a PAF conjugate, and the use of these compositions in the treatment, prophylaxis or prevention of cardiovascular disease, such as atherosclerosis, for example in the treatment, prevention or reduction of further progression of atherosclerosis. Furthermore, the invention also relates to the use of PAF derivatives, PAF conjugates or said antibody preparation, for example monoclonal antibody to produce a pharmaceutical composition optionally with an adjuvant.

Furthermore the invention relates to diagnosing the absence, presence and/or levels of antibodies, for example IgM, IgG or IgA antibodies, related to increased or decreased risk of developing cardiovascular diseases.

By PAF is meant 1-O-alkyl-2-acetyl-sn-glycero-3-phosphocholine according to the formula.

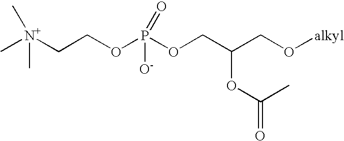

Preferably the alkyl group is a palmityl (hexadecyl) group or an oleyl (octadecyl) group.

By a PAF conjugate is meant a PAF moiety linked to a carrier, preferably via a spacer. The PAF moiety can be covalently or non-covalently linked to the carrier. The PAF moiety may be a derivative of PAF. Preferably the PAF moiety is linked to the carrier via the alkyl group.

Examples of derivatives of PAF are compounds according to the formula

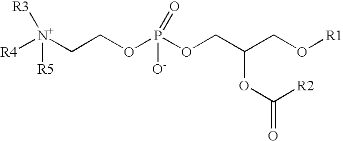

where R1 is C₂ to C₂₅ alkylene, alkenylene, or alkynylene linking group, and R2 to R5 are independently selected from C₁ to C₆ alkyl, as described in WO 87/05904 and U.S. Pat. No. 5,061,626,

compounds according to the formula

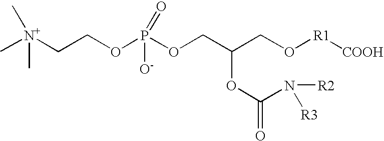

where R1 is C₂ to C₁₈ alkyl, preferably pentadecyl, R2 is H, methyl, ethyl, propyl, or isopropyl; R3 is methyl, ethyl, propyl or isopropyl, as described by Karasawa K et al. J Biochem (Tokyo) 1991, 110:683-687,
compounds according to the formula

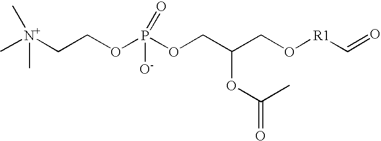

where R1 is C₆ to C₁₈ alkyl, preferably hexyl or dodecyl as described by SmaI MA et al. Lipids 1991, 26:1130-5,
compounds according to the formula

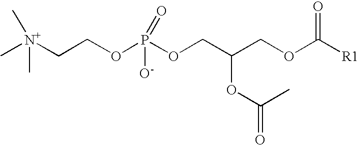

where R1 is C₆ to C₁₈ alkyl, preferably hexyl or dodecyl (1-acyl-PAF), as described by Muzya G I et al. Immunologiya (Moscow) 1997, 6, 9-11.

The carrier can be, for example, a protein, a carbohydrate, a polymer, latex beads, or colloid metal.

The PAF conjugate may for example be a protein-PAF conjugate, such as a human serum albumin (HSA)-PAF conjugate, a transferrin-PAF conjugate, a keyhole limpet hemocyanin (KLH)-PAF conjugate or a bovine serum albumin (BSA)-PAF conjugate.

Examples of PAF conjugates and generation of anti-PAF antibodies are described in WO 87/05904 and by Karasawa K et al. (Antibodies to synthetic platelet-activating factor (1-O-alkyl-2-O-acetyl-sn-glycero-3-phosphocholine) analogues with substituents at the sn-2 position. J Biochem (Tokyo). 1991, 110:683-7), Macpherson J L et al. (Production and characterization of antibodies to platelet-activating factor. J Lipid Mediat. 1992, 5:49-59), SmaI MA et al. (Synthesis of a PAF immunogen and production of PAF-specific antibodies. Lipids. 1991, 26:1130-5), Tomii A and Masugi F. (Production of anti-platelet-activating factor antibodies by the use of colloidal gold as carrier. Jpn J Med Sci Biol. 1991, 44:75-80), and Wang C and Tai H H. (A sensitive and specific radioimmunoassay for platelet-activating factor. Lipids. 1992, 27:206-8), and U.S. Pat. No. 5,061,626 the content of which is hereby incorporated by reference.

Cardiovascular diseases that can be treated and/or prevented according to invention are exemplified, but not limited to, atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty.

In the use according to the first aspect of the invention, or the method according to the second aspect of the invention, the cardiovascular disease may be selected from atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty.

Preferably, in the use according to the first aspect of the invention, or the method according to the second aspect of the invention, the medicament is for administration by injection.

The fifth aspect of the invention may provide methods to determine the presence or absence of antibodies, for example IgM, IgG or IgA antibodies, with reactivity to a PAF conjugate which are related to an increased or decreased risk of developing cardiovascular diseases.

Typically, the method of the fifth aspect of the invention comprises exposing the PAF conjugate to a sample from an individual and detecting antibodies which have bound to the PAF conjugate.

Preferably, in the method of the fifth aspect of the invention, the individual is a human.

Preferably, in the method of the fifth aspect of the invention, the sample is serum.

The use of the PAF conjugate according to the sixth aspect of the invention may correspond to the method of using the PAF conjugate according to the fifth aspect of the invention.

Preferably, in the fifth or sixth aspects of the invention, PAF is linked to a carrier via a spacer. In this embodiment, typically the carrier is a protein, preferably KLH (keyhole limpet hemocyanin), transferrin, human serum albumin (HSA) or bovine serum albumin (BSA). Alternatively, the carrier may be latex beads.

Typically, according to the fifth aspect of the invention, antibodies which have bound to the PAF conjugate are determined by an assay, preferably an immunoassay.

The patient's levels of antibodies, e.g., IgM, IgG or IgA antibodies, with reactivity to the PAF conjugate may be assessed using an immunoassay. Examples of suitable immunoassays are described below and will in any case be apparent to those skilled in the art.

Typically, in the fifth and sixth embodiments of the invention, low levels of antibodies with reactivity to the PAF conjugate are indicative of an increased risk of developing cardiovascular diseases. Conversely, high levels of antibodies with reactivity to the PAF conjugate are indicative of a reduced risk of developing cardiovascular diseases. Typically, antibodies are determined in a sample of patient plasma or serum.

In any given population, levels of antibodies with reactivity to a PAF conjugate are likely to vary. The level of antibodies with reactivity to a PAF conjugate determined for any given individual may be categorised as high or low by reference to the range observed in the wider population. For example, a level of such antibodies below a particular percentile value determined with reference to the wider population may be categorised as a low level. Suitably, a low level may correspond to a value below the 25^{th} percentile, or below the 20^{th}, 10^{th} or 5^{th} percentile. A high level may correspond to a value of above the 5^{th}, 10^{th}, 20^{th}, or 25^{th} percentile, for example.

Where an individual is characterised as possessing low levels of antibodies with reactivity to a PAF conjugate, this information may assist in the diagnosis or prognosis of increased risk of development or progression of ischemic cardiovascular disease. A clinician may take other factors into account in arriving at a diagnosis or prognosis. Where the individual is considered to have an increased risk of developing ischemic cardiovascular disease, prophylactic treatments and/or life-style changes may be recommended. Where the individual is diagnosed as having a progressive ischemic cardiovascular disease, his or her clinician may recommend treatments and/or life-style changes tailored to the individual.

In the fifth and sixth aspects of the invention, levels of antibodies may be characterised by assaying for all antibodies with reactivity to a PAF conjugate, or for only antibodies of a particular isotype, such as IgM, IgG or IgA, or for a combination of two or more antibody isotypes. Preferably, the level of IgG is determined.

Immunoassays can be competitive or noncompetitive. In a typical competitive immunoassay, the antibody in the sample competes with labeled antibody to bind with the PAF conjugate. The amount of labeled antibody bound to the PAF conjugate is then measured. There is an inverse relationship between concentration of antibody in the sample and the quantity of labeled antibody detected. In noncompetitive immunoassays, antibody in the sample is bound to the PAF conjugate, then a labeled detection reagent, typically an anti-immunoglobulin antibody, is bound to the antibody. The amount of labeled detection reagent bound to the antibody is then measured. Unlike the competitive method, the results of the noncompetitive method will be directly proportional to the concentration of the antibody.

In a noncompetitive immunoassay or western blot, a labeled detection reagent, typically an anti-immunoglobulin antibody, is used to detect antibody bound to the PAF conjugate. A suitable anti-immunoglobulin antibody must bind specifically to immunoglobulin of the species from which the sample is obtained. It may bind to all immunoglobulin isotypes of that species, or only a subset of isotypes. For example, it may bind only to IgA, IgD, IgE, IgG or IgM, or combinations of two or more of these isotypes. The anti-immunoglobulin antibody may bind specifically only to certain subtypes of any given isotype. Subtypes of human IgA are IgA1 and IgA2. The anti-immunoglobulin antibody may bind to one or both of these subtypes. Subtypes of human IgG are IgG1, IgG2, IgG3 and IgG4. The anti-immunoglobulin may bind to one or more of these human IgG subtypes. It will be appreciated that there are different isotypes and subtypes in different vertebrate species.

In radioimmunoassay, the antibody or detection reagent is labeled with a radioisotope, such as ¹³¹I or ¹²⁵I. In enzyme immunoassays, the antibody or detection reagent is labeled with an enzyme. Suitable enzymes are capable of being detected with the use of a chromogenic substrate. A chromogenic substrate is a substance which, as a result of the reaction with the enzyme, gives rise to a coloured product which can thus be detected spectrophotometrically. Enzymes such as horse radish peroxidase, alkaline phosphatase, beta-galactosidase, and pyrophosphatase from E. coli have been widely employed. Chemi-luminescent systems based on enzymes such as luciferase can also be used. Other labels include fluorescent labels such as fluorophores of the Alexa series.

Conjugation of the antibody or detection reagent with the vitamin biotin is frequently used since this can readily be detected by its reaction with enzyme- or fluorophore-linked avidin or streptavidin to which it binds with great specificity and affinity.

In a typical noncompetitive enzyme immunoassay, the sample to be analyzed is placed in contact and incubated with the PAF conjugate adsorbed on a solid substrate. Any anti-PAF conjugate antibodies that are possibly present in the sample are thus specifically bound by the PAF conjugate adsorbed on the solid substrate, producing a PAF conjugate/anti-PAF conjugate antibody complex. The sample is then separated from the solid substrate so as to eliminate non-bound materials, for example, by washing. In the next step of the method, an indicator antibody capable of binding any anti-PAF conjugate antibodies that are present on the substrate in the form of a PAF conjugate/anti-PAF conjugate antibody complex is added to the solid substrate, thus producing a PAF conjugate/anti-PAF conjugate antibody/indicator antibody complex. The indicator antibody may, for example, be an anti-human IgG immunoglobulin raised in a non-human animal species. Finally, the presence of the PAF conjugate/anti-PAF conjugate antibody/indicator antibody complex on the solid substrate is detected, the presence of said complex on the solid substrate being indicative of the presence of anti-PAF conjugate antibodies in the sample from the individual.

Typically, the solid substrate is a micro-titration plate, for example, of the type commonly used for performing ELISA immunological assays. The micro-titration plate is preferably a polystyrene plate. Other suitable solid substrates are latex particles, beads and coated red blood cells. Conveniently, the PAF conjugate is adsorbed to the solid substrate by incubating the PAF conjugate in a buffer with the solid substrate. Suitable buffers include carbonate buffer or phosphate buffered saline. Alternatively, the PAF conjugate may be covalently linked to the solid substrate. Typically, after adsorption or covalent linkage of the PAF conjugate to the solid substrate, the solid substrate is incubated with a blocking agent to reduce non-specific binding of matter from the sample to the solid substrate. Suitable blocking agents include bovine serum albumin.

It is preferred that a quantitative estimate of antibody which can bind to the PAF conjugate is obtained by one or more of the above techniques. In typical non-competitive assays, a linear relationship between the measured variable, whether it be optical density or some other read-out, and antibody concentration, is assumed. For example, if sample A has double the optical density of sample B in the assay (background having been subtracted from both), it is assumed that the concentration of antibody is double in A compared to B. However, it is preferable to construct a standard curve of serial dilutions of a pool of positive serum samples. Preferably, such dilutions are assayed at the same time as the test samples. By doing this, any variation from the linear relationship may be taken into account in determining the quantity of antibody in the samples.

It may be desirable to measure antibodies reactive with oxLDL or malondialdehyde modified LDL (MD-LDL) as well as measuring antibodies, e.g., IgM, IgG or IgA antibodies, with reactivity to the PAF conjugate. It may alternatively or in addition be desirable to measure levels of lipoprotein associated phospholipase A₂ (LpPLA₂), homocystein, C-reactive protein (CRP), HSP70, high density lipoprotein (HDL), TNF, in particular TNFα, and/or HSP60 as well as measuring antibodies, e.g., IgM, IgG or IgA antibodies, with reactivity to the PAF conjugate. Assaying for such factors may assist in the diagnosis or prognosis of increased risk of development or progression of cardiovascular disease.

It is contemplated that any method or composition described herein can be implemented with respect to any other method or composition described herein. Similarly, any embodiment discussed with respect to one aspect of the invention may be used in the context of any other aspect of the invention.

Throughout this application, the term “about” is used to indicate that a value includes the standard deviation of error for the device or method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

The examples disclosed below are provided only for the purpose of illustrating the present invention and should not be considered as any limitation of the scope as outlined in the appended claims. Document referred to herein are hereby incorporated by reference.

An example of a method to determine the absence or presence and/or level of IgG antibodies with reactivity to a PAF conjugate which is related to an increased or decreased risk of developing cardiovascular diseases is described. Other methods known in the art can also be used.

Similar methods may be used to determine the absence or presence and/or level of IgM or IgA antibodies with reactivity to a PAF conjugate.

A. PAF Conjugate

The following PAF-BSA conjugate was synthesized and used in methods according to the invention

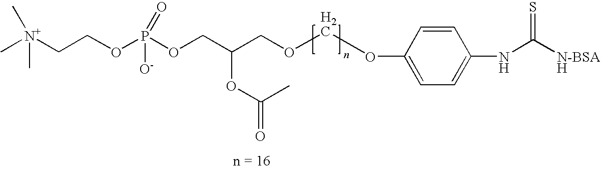

B. Methods to Determine the Absence or Presence and/or Level of Antibodies with Reactivity to a PAF Conjugate

A microtitre plate was coated with 5 μg/ml PAF-BSA conjugate (described above), in phosphate buffered saline (PBS).

After washings with PBS, the plates were blocked with a 1% BSA solution. Serum samples were diluted (1:101) in sample diluent and added to the plates. The plates were incubated for 30 minutes at room temperature and washed. Horse radish peroxidase conjugated rabbit anti-human IgG diluted 1:1000 was added and incubated at room temperature for 30 minutes. After washings, colour was developed by adding 3,3′,5,5′, tetramethyl benzidine (TMB) substrate and the plates were incubated for 10 minutes at room temperature in the dark. The absorbance was read in a spectrophotometer at 450 nm. The levels of antibodies reactive with PAF-BSA conjugate was calculated as the ratio between the absorbance obtained from the tested sample and the absorbance obtained from a positive control included in each assay.

C. Antibodies Against PAF Conjugates

Antibodies against a PAF conjugate can be produced as described above. See, e.g., WO 87/05904, Macpherson J L et al. (Production and characterization of antibodies to platelet-activating factor. J Lipid Mediat. 1992, 5:49-59), and SmaI MA et al. (Synthesis of a PAF immunogen and production of PAF-specific antibodies. Lipids. 1991, 26:1130-5).

D. Monoclonal Antibodies Against a PAF Conjugate

Monoclonal antibodies against PAF can be produced using any standard method known in the art. See for example Tomii A and Masugi F. (Production of anti-platelet-activating factor antibodies by the use of colloidal gold as carrier. Jpn J Med Sci Biol. 1991, 44:75-80).

Other antibodies reactive with PAF conjugate can be prepared using methods well known to those skilled in the art. For example, a subfraction of a human immunoglobulin preparation with reactivity to a PAF conjugate can be prepared, for example as described below, for example by affinity purification using a PAF conjugate.

Intravenous immunoglobulin preparations (e.g., IGIV; Baxter and others) is a highly purified preparation of IgG commercially available and is used in the treatment of patients who have no, or very low levels of antibody production. Immunoglobulin preparations include those available from the following manufacturers: Baxter (US), e.g., Gammagard®, Isiven (Antimo Naples, Italy), Omrix (Tel-Hashomer, Israel), Miles (Biological Products Division, West Heaven, Conn.), Sclavo (Lucca, Italy), Sandoz (Novartis, Basel, Switzerland), e.g., Sandoglobulin®, Biotest Diagnostic Corporation (Deville, N.J.). Examples of immunoglobulin preparations are GammagardS/D®, GammarlV®, Gaimnar-PIV®, Gammimune N®, Iveegam®, Panglobulin®, Polygam S/D®, Sandoglobulin®, Venoglobulin®. Immunoglobulin preparations typically contain some IgM as well as IgG. Trace amounts of IgM are present in Gammagard®. Pentaglobin (Biotest) is an enriched IgM preparation which has been used for treatment of SARS. The subfraction with reactivity to a PAF conjugate may comprise both IgG and IgM, or may be selected to comprise mainly IgG (for example by starting with an IgG-rich preparation such as Gammagard® and/or by selecting for IgG); or mainly IgM (for example by starting with an IgM-rich preparation such as Pentaglobin and/or by selecting for IgM).

E. Active Immunization

One embodiment of the present invention is thus to use a PAF derivative or PAF conjugate for the preparation of a pharmaceutical composition to be used in the treatment, prophylaxis or prevention of cardiovascular disease, such as atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty. The conjugate can be PAF or PAF derivative linked to a pharmaceutically acceptable protein, carbohydrate, or polymer. The pharmaceutical composition is preferably given by injection.

The proposed method of active immunization will modulate the antibody titre which in turn will have a positive effect on the development of cardiovascular disease.

F. Passive Immunization

Another embodiment of the invention is to use an antibody preparation, for example a monoclonal antibody, recognizing a PAF conjugate for the preparation of a pharmaceutical composition to be used in the treatment, prophylaxis or prevention of cardiovascular disease, such as atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty. The monoclonal antibody can be produced using methods known in the art.

G. Diagnosis and Risk Assessment

A further embodiment of the invention is to provide a method of diagnosing the absence, presence and/or levels of antibodies, for example IgA, IgM or IgG antibodies, with reactivity towards a PAF-conjugate which factor is related to an increased or decreased risk of developing cardiovascular diseases, using a PAF conjugate. A preferred method is an immunoassay. The method may be used in assessing the patient's risk of developing or progression of cardiovascular disease.

### Example 1

The following example is included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Subjects

In an observational study from Malmö (the Malmö Diet and Cancer Study), about 6000 out of 30000 subjects from the cohort were recruited for extensive cardiovascular investigations, including non-invasive assessment of subclinical atherosclerosis through ultrasound measurements of the carotids. There were in total 137 CVD cases (mainly myocardial infarction (MI) and ischemic stroke) and 384 age and sex-matched controls. The cutoff level for anti-PAF-BSA was 307 for the tenth percentile of antiPAF levels. The cut-off level for anti-PAF-BSA IgG was 60.8 for the 25^{th} percentile. There were in total 58 CVD cases with anti-PAF-BSA IgG levels below the 25^{th} percentile (42%), and 79 controls (21%), corresponding to a relative hazard of 3.19 (95% CI 2.04-4.99) (Table 1).

The results suggest that low anti-PAF-BSA levels are predictive for the occurrence of cardiovascular disease in healthy subjects, and could act as markers for cardiovascular diseases.

B. Reagents

Maxisorp lock well, C96, microtiter plates were purchased from Nunc (Roskilde Denmark), PAF-BSA was synthesized and provided by IsoSep, Uppsala (Sweden).

Horseradish peroxidase conjugated to rabbit anti-human IgG (y-specific) was purchased from DakoCytomation, Stockholm (Sweden). TMB substrate was purchased from Phadia, Freiburg (Germany).

C. Determination of Antibodies Against PAF-Conjugate

IgG antibodies to PAF-BSA were determined by enzyme-linked immunosorbent assay (ELISA). One serum yielding an absorbance of 0.5-0.7 OD was used as an internal standard and tested on every plate. The plateau of antibody binding was reached with the antigen concentration of 5 μg/ml. C96 microtiter maxsorp plates were therefore coated with PAF-BSA (5 μg/ml) 100 μg/well in PBS and incubated overnight at 4° C. After aspiration of the coating solution, the plates were blocked with 1% BSA-PBS for 2 h at room temperature. The blocking solution was aspirated and the serum samples diluted 1:101 in 0.2% BSA-PBS were added at 100 μl/well. Plates were incubated for 30 minutes at room temperature, as described above, and washed three times with washing buffer (Phadia, Freiburg, Germany). One hundred μl horseradish conjugated rabbit anti-human IgG (diluted 1:1000) was added per well and incubated at room temperature for 30 minutes. After washing three times, as described above, colour was developed by the addition of 100 μl/well of TMB substrate. After 10 minutes incubation at room temperature in the dark, the reaction was stopped by the addition of 0.5 M H₂SC₄ at 50 μl/well. The plates were read in an ELISA DigiScan spectrophotometer at 450 nm. All samples were measured in duplicates and the coefficient of variation was below 15% for all samples.

D. Results

TABLE 1

| percentiles | P | RR | 95% CI |
|---|---|---|---|
| 25 | 0.0001 | 3.190 | 2.039-4.992 |
| 20 | 0.0001 | 3.085 | 1.929-4.933 |
| 15 | 0.0001 | 2.780 | 1.667-4.636 |
| 10 | 0.0001 | 3.294 | 1.798-6.034 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-4. (canceled)

5. A method for immunization and prophylaxis, prevention and treatment of a mammal, including a human, against a cardiovascular disease, comprising the step of administering to the mammal a pharmaceutical composition comprising at least one PAF derivative or PAF conjugate, so as to raise the antibody titre for the at least one PAF derivative or PAF conjugate in the mammal.

6. The method of claim 5, wherein the pharmaceutical composition is administered in combination with an adjuvant.

7. A method for immunization and prophylaxis, prevention and treatment of a mammal, including a human, against a cardiovascular disease comprising the step of administering to the mammal a pharmaceutical composition comprising an antibody preparation, for example a monoclonal antibody, with reactivity to a PAF conjugate.

8. The method according to claim 5, wherein the cardiovascular disease is selected from the group consisting of atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty.

9. The method of claim 5, wherein the composition is administered by injection.

10. A method of diagnosing the presence or absence of IgM, IgG or IgA antibodies in a mammal related to increased or decreased risk of development or progression of ischemic cardiovascular diseases by measuring binding of antibodies to a PAF conjugate, wherein low levels of the antibodies with reactivity to the PAF conjugate are indicative of an increased risk of development or progression of ischemic cardiovascular diseases.

11. The method of claim 10, comprising exposing the PAF conjugate to a sample from the mammal and detecting antibodies which have bound to the PAF conjugate.

12. The method of claim 11, wherein the mammal is a human.

13. The method of claim 11, wherein the sample is serum.

14. The method of claim 10, wherein PAF is linked to a carrier via a spacer.

15. The method of claim 11, wherein the carrier is a protein.

16. The method of claim 15, wherein the protein is KLH (keyhole limpet hemocyanin), transferrin, human serum albumin (HSA) or bovine serum albumin (BSA).

17. The method of claim 14, wherein the carrier is a latex bead.

18. The method of claim 10, wherein antibodies bound to the PAF conjugate are determined by an immunoassay.

19. The method of claim 5, wherein the PAF derivative or the PAF of the PAF conjugate is a compound according to the formula:
(a) 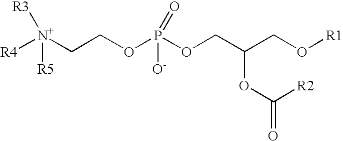
where R1 is C₂ to C₂₅ alkylene, alkenylene, or alkynylene linking group, and R2 to R5 are independently selected from C₁ to C₆ alkyl;
(b) 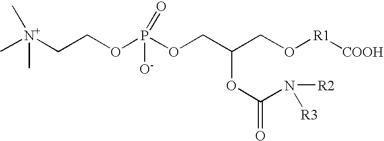
where R1 is C₂ to C₁₈ alkyl, preferably pentadecyl, R2 is H, methyl, ethyl, propyl, or isopropyl; R3 is methyl, ethyl, propyl or isopropyl;
(c) 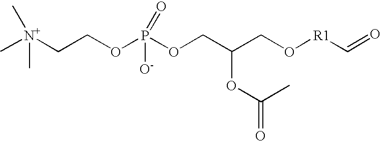
where R1 is C₆ to C₁₈ alkyl, preferably hexyl or dodecyl; or
(d) 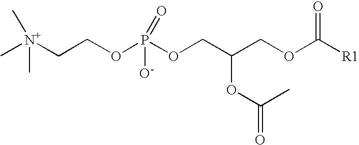
where R1 is C₆ to C₁₈ alkyl, preferably hexyl or dodecyl (1-acyl-PAF).

20. The method according to claim 7, wherein the cardiovascular disease is selected from the group consisting of atherosclerosis, acute myocardial infarction, stable and unstable angina, stroke, restenosis following artery grafting, artery stenting, and balloon angioplasty, especially restenosis following coronary artery bypass grafting, coronary artery stenting and coronary angioplasty.

21. The method of claim 7, wherein the composition is administered by injection.

22. The method of claim 7, wherein the pharmaceutical composition is administered in combination with an adjuvant.

23. The method of claim 7, wherein the PAF of the PAF conjugate is a compound according to the formula:
(a) 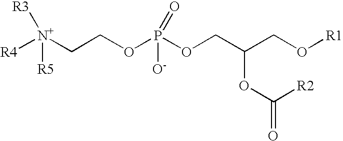
where R1 is C₂ to C₂₅ alkylene, alkenylene, or alkynylene linking group, and R2 to R5 are independently selected from C₁ to C₆ alkyl;
(b) 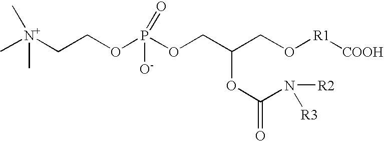
where R1 is C₂ to C₁₈ alkyl, preferably pentadecyl, R2 is H, methyl, ethyl, propyl, or isopropyl; R3 is methyl, ethyl, propyl or isopropyl;
(c) 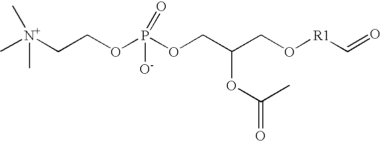
where R1 is C₆ to C₁₈ alkyl, preferably hexyl or dodecyl; or
(d) 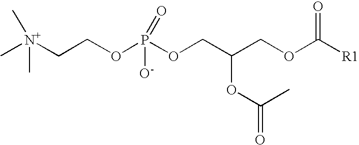
where R1 is C₆ to C₁₈ alkyl, preferably hexyl or dodecyl (1-acyl-PAF).

24. The method of claim 10, wherein the PAF of the PAF conjugate is a compound according to the formula:
(a) 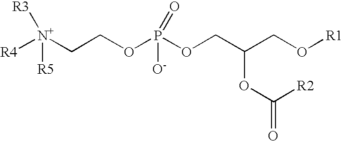
where R1 is C₂ to C₂₅ alkylene, alkenylene, or alkynylene linking group, and R2 to R5 are independently selected from C₁ to C₆ alkyl;
(b) 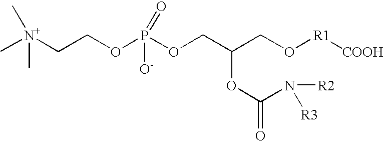
where R1 is C₂ to C₁₈ alkyl, preferably pentadecyl, R2 is H, methyl, ethyl, propyl, or isopropyl; R3 is methyl, ethyl, propyl or isopropyl;
(c) 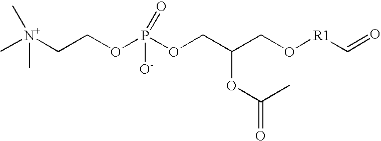
where R1 is C₆ to C₁₈ alkyl, preferably hexyl or dodecyl; or
(d) 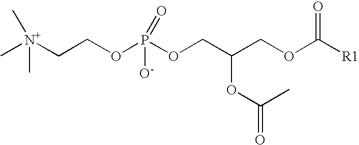
where R1 is C₆ to C₁₈ alkyl, preferably hexyl or dodecyl (1-acyl-PAF).
